(19) United States
(12) Patent Application Publication
(10) Pub. No.: US 2026/0116909 A1
(43) Pub. Date: Apr. 30, 2026

(54) LINKER AND SOLID PHASE CARRIER FOR NUCLEIC ACID SYNTHESIS

(71) Applicant: FUJIFILM Wako Pure Chemical Corporation, Osaka-shi, Osaka (JP)

(72) Inventors: Yoshiyuki HARI, Tokushima (JP); Yasufumi FUCHI, Tokushima (JP); Kazuki YAMAMOTO, Tokushima (JP); Yuta ITO, Tokushima (JP)

(21) Appl. No.: 18/995,532

(22) PCT Filed: Jul. 21, 2023
(86) PCT No.: PCT/JP2023/026790

§ 371 Date: Jan. 16, 2025

(30) Foreign Application Priority Data
Jul. 29, 2022 (JP) 2022-121221

(51) Int. Cl. C07H 21/04; C07D 493/08

(52) U.S. Cl. CPC C07H 21/04; C07D 493/08

(57) ABSTRACT

An object of the present invention is to provide a solid-phase carrier for nucleic acid synthesis, in which a nucleic acid and an adduct can be easily separated by HPLC in a case where the nucleic acid is synthesized using the solid-phase carrier for nucleic acid synthesis and the nucleic acid is cut out, a compound which serves as a linker of the solid-phase carrier, a compound which serves as a precursor of the linker, and a method for producing a nucleic acid using the solid-phase carrier. The compound according to the embodiment of the present invention is a compound represented by the following general formula (1).

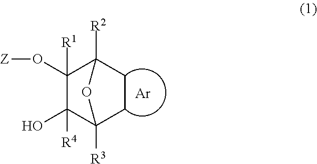

In the general formula (1), Ar represents a tri- to tetracyclic aromatic ring which may have a substituent, Z represents a hydrogen atom or a protective group eliminable by an acid, and R¹ to R⁴ each independently represent a hydrogen atom, an alkyl group, or an alkoxy group.

### BACKGROUND OF THE INVENTION

### 1. Field of the Invention

The present invention relates to a compound, a linker, a solid-phase carrier, and a method for producing a nucleic acid.

### 2. Description of the Related Art

In the related art, a solid-phase carrier having a 1,2-diol unit, which is called a universal linker (hereinafter, also referred to as “universal support”), has been used for the synthesis of a nucleic acid (for example, Patent Literature 1, Non-Patent Literatures 1 to 2). The use of the universal support has an advantage that it is not necessary to prepare a solid-phase carrier corresponding to the 3′-terminal of the nucleic acid.

### CITATION LIST

### Patent Literature

Patent Literature 1: WO2004/0152905A

### Non-Patent Literatures

Non-Patent Literature 1: Synthesis, 2021, 53, 4440-4448
Non-Patent Literature 2: Tetrahedron, 2021, 92, 132261

### SUMMARY OF THE INVENTION

### Technical Problems

In a case where a nucleic acid is synthesized using a universal support, it is necessary to cut out the nucleic acid from the universal support by treatment with an ammonia aqueous solution or the like. For example, as shown in the following scheme (here, CPG: controlled pore glass, Nu⁻: nucleophilic agent, R: hydrogen atom or substituent), a nucleic acid is cut out.

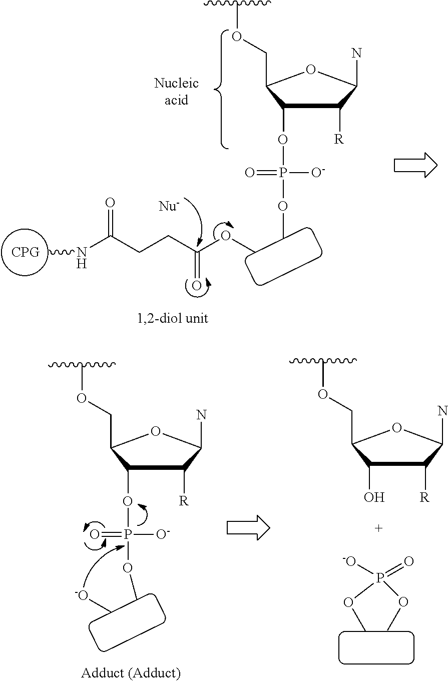

In this case, since there is a case that an adduct in which the 1,2-diol unit is added to the nucleic acid may remain, in such a case, purification by high performance liquid chromatography (HPLC) or the like is necessary.

In the meantime, in a case where by the present inventors, a nucleic acid is synthesized using the universal support described in WO2004/0152905A, and the nucleic acid is cut out, it has been found that a peak of the nucleic acid and a peak of an adduct may not be sufficiently separated by HPLC.

In view of the circumstances, an object of the present invention is to provide a solid-phase carrier for nucleic acid synthesis, in which a nucleic acid and an adduct can be easily separated by HPLC in a case where the nucleic acid is synthesized using the solid-phase carrier for nucleic acid synthesis and the nucleic acid is cut out, a compound which serves as a linker of the solid-phase carrier, a compound which serves as a precursor of the linker, and a method for producing a nucleic acid using the solid-phase carrier.

### Solution to Problems

As a result of intensive studies on the problems, the present inventors have found that the object can be achieved by using a specific compound having a tri- to tetracyclic aromatic ring which is high lipophilic, and have completed the present invention.

That is, the present inventors have found that the object can be achieved by the following configurations.

(1) A compound represented by the general formula (1) described later.

(2) A compound represented by the general formula (2) described later.

(3) The compound according to (1) or (2), in which, in the general formula (1) or (2) described later, Ar represents an anthracene ring, a phenanthrene ring, a tetracene ring, or a pyrene ring, each of which may have a substituent.

(4) The compound according to any one of (1) to (3), in which, in the general formula (1) or (2) described later, Ar represents a phenanthrene ring which may have a substituent.

(5) The compound according to any one of (1) to (4), in which, in the general formula (1) or (2) described later, Z represents a protective group eliminable by an acid.

(6) The compound according to (5), in which the protective group eliminable by an acid is a trityl-based protective group or a silyl-based protective group.

(7) The compound according to any one of (1) to (6), in which, in the general formula (1) or (2) described later, R¹ to R⁴ each represent a hydrogen atom.

(8) The compound according to (2) and according to any one of (3) to (7) that depend from at least (2), in which, in the general formula (2) described later, L represents an alkylene group which may have an oxygen atom, an arylene group which may have an oxygen atom, or a combination of the alkylene group and the arylene group.

(9) A linker of a solid-phase carrier for nucleic acid synthesis, obtained by using the compound according to any one of (1) to (8).

(10) A solid-phase carrier formed of a compound represented by the general formula (3) described later.

(11) The solid-phase carrier according to (10), in which the solid-phase carrier is for nucleic acid synthesis.

(12) The solid-phase carrier according to (10) or (11), in which, in the general formula (3) described later, Ar represents an anthracene ring, a phenanthrene ring, a tetracene ring, or a pyrene ring, each of which may have a substituent.

(13) The solid-phase carrier according to any one of (10) to (12), in which, in the general formula (3) described later, Ar represents a phenanthrene ring which may have a substituent.

(14) The solid-phase carrier according to any one of (10) to (13), in which, in the general formula (3) described later, Z represents a protective group eliminable by an acid.

(15) The solid-phase carrier according to (14), in which the protective group eliminable by an acid is a trityl-based protective group or a silyl-based protective group.

(16) The solid-phase carrier according to any one of (10) to (15), in which, in the general formula (3) described later, R¹ to R⁴ each represent a hydrogen atom.

(17) The solid-phase carrier according to any one of (10) to (16), in which, in the general formula (3) described later, L represents an alkylene group which may have an oxygen atom, an arylene group which may have an oxygen atom, or a combination of the alkylene group and the arylene group.

(18) The solid-phase carrier according to any one of (10) to (17), in which, in the general formula (3) described later, Sp represents a porous polymer carrier or a glass-based porous carrier.

(19) A method for producing a nucleic acid, comprising a step of performing a nucleic acid synthesis reaction on the solid-phase carrier according to (10) to (18).

(20) The method for producing a nucleic acid according to (19), in which the nucleic acid synthesis reaction is performed by a phosphoramidite method.

### Advantageous Effects of Invention

As shown below, according to the present invention, it is possible to provide a solid-phase carrier for nucleic acid synthesis, in which a nucleic acid and an adduct can be easily separated by HPLC in a case where the nucleic acid is synthesized using the solid-phase carrier for nucleic acid synthesis and the nucleic acid is cut out, a compound which serves as a linker of the solid-phase carrier, a compound which serves as a precursor of the linker, and a method for producing a nucleic acid using the solid-phase carrier. In addition, the solid-phase carrier according to the embodiment of the present invention also has an effect that, in a case where a nucleic acid is synthesized using the solid-phase carrier and the nucleic acid is cut out, a peak of a cyclic phosphate derived from the linker is observed by HPLC. In addition, the solid-phase carrier according to the embodiment of the present invention also has an effect of being difficult to be decomposed because the solid-phase carrier is formed of a stable structure.

### BRIEF DESCRIPTION OF THE DRAWINGS

FIG. 1 is an HPLC chart of Example 1 and Comparative Example 1.

FIG. 2 is an HPLC chart of Example 1 and Comparative Example 1.

FIG. 3 is an HPLC chart of Example 2.

FIG. 4 is an HPLC chart of Example 3.

FIG. 5 is an HPLC chart of Example 4.

### DESCRIPTION OF THE PREFERRED EMBODIMENTS

Hereinafter, the compound, linker, solid-phase carrier, and method for producing a nucleic acid according to the embodiment of the present invention will be described.

It should be noted that, in the present specification, a numerical range represented by “to” means a range including numerical values before and after “to” as a lower limit value and an upper limit value.

In addition, in the present specification, adenine may be represented by “A”, guanine may be represented by “G”, cytosine may be represented by “C”, and thymine may be represented by “T”.

In addition, facts that, in a case where a nucleic acid is cut out, the nucleic acid and the adduct can be easily separated by HPLC, a peak of a cyclic phosphate derived from the linker can be easily observed by HPLC, and the nucleic acid is difficult to be decomposed, may be collectively referred to as “the effects of the present invention are excellent”.

### [1] Compound (1)

A compound (1) according to the embodiment of the present invention is a compound represented by the following general formula (1).

The compound (1) according to the embodiment of the present invention is a compound suitable as a precursor of a linker of a solid-phase carrier according to the embodiment of the present invention, which will be described later. Hereinafter, the compound (1) according to the embodiment of the present invention is also referred to as “specific linker precursor”.

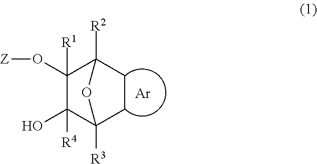

In the general formula (1), Ar represents a tri- to tetracyclic aromatic ring which may have a substituent, Z represents a hydrogen atom or a protective group eliminable by an acid, and R¹ to R⁴ each independently represent a hydrogen atom, an alkyl group, or an alkoxy group.

### [Ar]

As described above, in the general formula (1), Ar represents a tri- to tetracyclic aromatic ring which may have a substituent.

Here, the tri- to tetracyclic aromatic ring means a ring in which three or four monocyclic aromatic rings are fused. In addition, the aromatic ring is a ring in which the number of electrons included in the π-electron system is 4n+2 (n is an integer of 0 or more). From the reason that the effect of the present invention is more excellent, the monocyclic aromatic ring is preferably a benzene ring. Hereinafter, the “tri- to tetracyclic aromatic ring” is also referred to as a “specific aromatic ring”.

Specific examples of the specific aromatic ring include an anthracene ring, a phenanthrene ring (phenanthrene ring), a chrysene ring, a pyrene ring, a triphenylene ring, a tetracene ring, and the like. Among these, from the reason that the effect of the present invention is more excellent, the anthracene ring, the phenanthrene ring, the tetracene ring, or the pyrene ring is preferable and the phenanthrene ring is more preferable.

The substituent which may be included in the specific aromatic ring is not particularly limited, and specific examples thereof include a substituent W described later.

From the reason that the effect of the present invention is more excellent, the substituent is preferably an alkyl group, an alkoxy group, a dialkylamino group, or a halogeno group (halogen atom).

### [Z]

As described above, Z represents a hydrogen atom or a protective group eliminable by an acid. Among these, from the reason that the effect of the present invention is more excellent, the protective group eliminable by an acid is preferable.

The protective group eliminable by an acid is not particularly limited, but from the reason that the effect of the present invention is more excellent, a protective group eliminable by a Brönsted acid such as trichloroacetic acid or dichloroacetic acid is preferable, a trityl-based protective group or a silyl-based protective group is more preferable, and the trityl-based protective group is still more preferable.

Examples of the trityl-based protective group include a trityl group which may be substituted with any substituent (for example, a substituent selected from a C₁₋₆ alkoxy group, a C₁₋₆ alkyl group, a halogen atom, and the like (two or more substituents may be combined to form a ring)), and specific examples thereof include a trityl group (a triphenylmethyl group (Tr)), a monomethoxytrityl group (for example, a 4-methoxyphenyldiphenylmethyl group (MMTr)), a dimethoxytrityl group (for example, a 4,4′-dimethoxyphenylphenylmethyl group (DMTr)), a 9-phenylxanthen-9-yl group (a pixyl group), and the like. From the reason that the effect of the present invention is more excellent, the trityl-based protective group is preferably the 4,4′-dimethoxyphenylphenylmethyl group (DMTr).

Examples of the silyl-based protective group include a silyl group tri-substituted with any substituent (for example, a substituent selected from a C₁₋₆ alkoxy group, a C₁₋₆ alkyl group, a phenyl group, and the like), and specific examples thereof include a trimethylsilyl group, a triethylsilyl group, an isopropyldimethylsilyl group, a tert-butyldimethylsilyl group, a dimethylmethoxysilyl group, a methyldimethoxysilyl group, a tert-butyldiphenylsilyl group, and the like. From the reason that the effect of the present invention is more excellent, the silyl-based protective group is preferably the trimethylsilyl group.

It should be noted that, in the present specification, examples of the “C₁₋₆ alkyl group” include linear, branched, or cyclic alkyl groups such as a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a tert-pentyl group, a cyclopentyl group, a hexyl group, and a cyclohexyl group. Among these, from the reason that the effect of the present invention is more excellent, the methyl group or the ethyl group is preferable.

In addition, in the present specification, examples of the “C₁₋₆ alkoxy group” include linear, branched, or cyclic alkoxy groups such as a methoxy group, an ethoxy group, an n-propoxy group, an isopropoxy group, an n-butoxy group, an isobutoxy group, a sec-butoxy group, a tert-butoxy group, an n-pentyloxy group, an isopentyloxy group, a sec-pentyloxy group, a tert-pentyloxy group, a cyclopentyloxy group, a hexyloxy group, and a cyclohexyloxy group. Among these, from the reason that the effect of the present invention is more excellent, the methoxy group or the tert-butoxy group is preferable.

In addition, in the present specification, examples of the “halogen atom” include a fluorine atom, a chlorine atom, a bromine atom, and an iodine atom. Among these, from the reason that the effect of the present invention is more excellent, the fluorine atom, the chlorine atom, or the bromine atom is preferable.

[R¹ to R⁴]

As described above, in the general formula (1), R¹ to R⁴ each independently represent a hydrogen atom, an alkyl group (for example, having 1 to 10 carbon atoms, preferably having 1 to 6 carbon atoms, and more preferably having 1 to 4 carbon atoms), or an alkoxy group (for example, having 1 to 10 carbon atoms, preferably having 1 to 6 carbon atoms, and more preferably having 1 to 4 carbon atoms).

From the reason that the effect of the present invention is more excellent, R¹ to R⁴ are preferably a hydrogen atom.

### [Substituent W]

The substituent W in the present specification will be described.

Examples of the substituent W include a halogeno group (halogen atom), an alkyl group (for example, a tert-butyl group) (including a cycloalkyl group, a bicycloalkyl group, and a tricycloalkyl group), an alkenyl group (including a cycloalkenyl group and a bicycloalkenyl group), an alkynyl group, an aryl group, a heterocyclic group (also referred to as a heterocyclic group), a cyano group, a hydroxy group, a nitro group, a carboxy group, an alkoxy group, an aryloxy group, a silyloxy group, a heterocyclic oxy group, an acyloxy group, a carbamoyl group, a carbamoyloxy group, an alkoxycarbonyloxy group, an aryloxycarbonyloxy group, an amino group (including an anilino group), an ammonio group, a dialkylamino group, an acylamino group, an aminocarbonylamino group, an alkoxycarbonylamino group, an aryloxycarbonylamino group, a sulfamoylamino group, an alkyl or arylsulfonylamino group, a mercapto group, an alkylthio group, an arylthio group, a heterocyclic thio group, a sulfamoyl group, a sulfo group, an alkyl or arylsulfinyl group, an alkyl or arylsulfonyl group, an acyl group, an aryloxycarbonyl group, an alkoxycarbonyl group, an aryl or heterocyclic azo group, an imide group, a phosphino group, a phosphinyl group, a phosphinyloxy group, a phosphinylamino group, a phosphono group, a silyl group, a hydrazino group, a ureido group, a boronate group (—B(OH)₂), a phosphate group (—OPO(OH)₂), a sulfate group (—OSO₃H), other known substituents, and the like.

### [Producing Method]

A method for producing the specific linker precursor is not particularly limited, and the specific linker precursor can be produced by combining known methods. For example, the specific linker precursor can be produced with reference to a synthesis path of a compound 1 or a compound 2 of Example 1 described later.

Specifically, for example, a precursor compound of the compound 1 is obtained by reacting a tri- to tetracyclic aromatic ring compound having a substituent W such as bromophenanthrene, and a halogeno group, with a furan which may have a substituent in the presence of a strong base such as sodium amide. Next, the compound 1 can be produced by converting the olefin into a diol with an oxidizing agent such as osmium tetroxide. In addition, the specific linker precursor (a compound in which Z is a protective group eliminable by an acid among compounds represented by the general formula (1)) can be produced by protecting one hydroxy group of the compound 1 with a protective group eliminable by an acid.

### [2] Compound (2)

A compound (2) according to the embodiment of the present invention is a compound represented by the following general formula (2).

The compound (2) according to the embodiment of the present invention is a compound suitable as a linker of a solid-phase carrier according to the embodiment of the present invention, which will be described later. Hereinafter, the compound (2) according to the embodiment of the present invention is also referred to as “specific linker”.

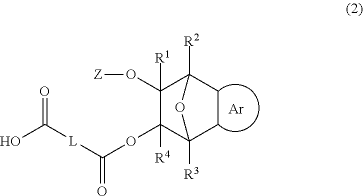

In the general formula (2), Ar represents a tri- to tetracyclic aromatic ring which may have a substituent, Z represents a hydrogen atom or a protective group eliminable by an acid, R¹ to R⁴ each independently represent a hydrogen atom, an alkyl group, or an alkoxy group, and L represents a divalent hydrocarbon group which may have an oxygen atom.

Specific examples and suitable aspects of Ar, Z, and R¹ to R⁴ are the same as those for the general formula (1).

### [L]

As described above, in the general formula (2), L represents a divalent hydrocarbon group which may have an oxygen atom.

The divalent hydrocarbon group is not particularly limited, and specific examples thereof include a divalent alkylene group (for example, having 1 to 10 carbon atoms), a divalent aromatic hydrocarbon group, and the like.

From the reason that the effect of the present invention is more excellent, L is preferably an alkylene group which may have an oxygen atom, an arylene group which may have an oxygen atom, or a combination of the alkylene group and the arylene group, more preferably a divalent alkylene group, and still more preferably an ethylene group.

### [Producing Method]

A method for producing the specific linker is not particularly limited, and the specific linker can be produced by combining known methods. For example, the specific linker can be produced with reference to a synthesis path of a compound 3 of Example 1 described later.

Specifically, for example, the specific linker can be produced by reacting a carboxylic acid anhydride such as succinic anhydride with the compound 2 in the presence of a base.

### [3] Solid-Phase Carrier

The solid-phase carrier according to the embodiment of the present invention is a solid-phase carrier formed of a compound represented by the general formula (3) (hereinafter, also referred to as “specific carrier”).

The specific carrier is suitably used for a solid-phase carrier for nucleic acid synthesis (universal support).

It should be noted that the compound (specific carrier) represented by the general formula (3) may be a compound in which one group (residue) obtained by removing Sp from the general formula (3) is bonded to Sp, or may be a compound in which a plurality of the residues are bonded to Sp.

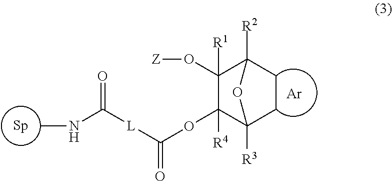

In the general formula (3), Ar represents a tri- to tetracyclic aromatic ring which may have a substituent, Z represents a hydrogen atom or a protective group eliminable by an acid, R¹ to R⁴ each independently represent a hydrogen atom, an alkyl group, or an alkoxy group, L represents a divalent hydrocarbon group, and Sp represents a solid-phase carrier.

Specific examples and suitable aspects of Ar, Z, R¹ to R⁴, and L are the same as those for the general formula (1) and the general formula (2).

### [Sp]

As described above, in the general formula (3), Sp represents a solid-phase carrier. Hereinafter, the solid-phase carrier represented by Sp is also referred to as a “Sp carrier”. The Sp carrier may have a spacer (for example, an alkylene group) for bonding to the carboxy group of the specific linker.

The Sp carrier is preferably a carrier for solid-phase synthesis, in which the reagents used in excess during nucleic acid synthesis can be easily removed by washing, and specific examples thereof include a glass-based porous carrier, a porous polymer carrier such as a polystyrene-based porous carrier and an acrylamide-based porous carrier, and the like.

From the reason that the effect of the present invention is more excellent, the Sp carrier is preferably the glass-based porous carrier or the porous polymer carrier (particularly, the polystyrene-based porous carrier).

The glass-based porous carrier refers to a porous carrier containing glass as a constituent component, and examples thereof include porous glass particles having a particle shape (CPG), but the glass-based porous carrier is not limited thereto. In a case of synthesizing the long-chain nucleotide, a pore size of CPG to be used is preferably 20 to 400 nm, more preferably 50 to 200 nm, and still more preferably 100 nm.

The polystyrene-based porous carrier is a porous carrier formed of a resin mainly constituted of a structural unit of styrene or a derivative thereof.

Examples of the polystyrene-based porous carrier include a porous carrier formed of styrene-hydroxystyrene-divinylbenzene copolymer particles (see JP2005-097545A, JP2005-325272A, and JP2006-342245A), a porous carrier formed of a styrene-(meth)acrylonitrile-hydroxystyrene-divinylbenzene copolymer (see JP2008-074979A), and the like.

The acrylamide-based porous carrier is a porous carrier formed of a resin mainly constituted of a structural unit of acrylamide or a derivative thereof.

Examples of the acrylamide-based porous carrier include a porous carrier formed of an aromatic monovinyl compound-divinyl compound-(meth)acrylamide derivative copolymer, and the like. In a case where the Sp carrier is an acrylamide-based solid-phase carrier, in a case where the content of the structural unit derived from the (meth)acrylamide derivative monomer is too small, the effect of avoiding the decrease in the amount of the synthesized nucleic acid and the decrease in the synthesis purity cannot be obtained, and on the other hand, in a case where the content thereof is too large, it is difficult to form a porous resin. Therefore, the content thereof is preferably 0.3 to 4 mmol/g, more preferably 0.4 to 3.5 mmol/g, and still more preferably 0.6 to 3 mmol/g.

A shape of the Sp carrier is not particularly limited, and may be any shape such as a flat plate shape, a particle shape, or a fiber shape, but from the viewpoint that the filling efficiency in the synthetic reaction container can be increased and the synthetic reaction container is less likely to be damaged, the carrier is preferably a carrier having a particle shape. In the present specification, the “particles” do not mean that the particles have a strictly spherical shape, and mean that the particles need only have a certain shape (for example, a substantially spherical shape such as an ellipsoidal shape, a polyhedral shape, a cylindrical shape, an irregular shape such as confetti shape, or the like).

A size (volume) of the Sp carrier is not particularly limited, but in a case where the average particle diameter measured by laser diffraction (scattering type) of porous particles is smaller than 1 μm, a problem occurs in that the back pressure is too high or the liquid feeding rate is too slow in a case of filling the carrier in a column to use, and on the other hand, in a case where the average particle diameter is larger than 1,000 μm, the voids between the carrier particles are increased and it is difficult to efficiently fill the carrier particles in a column having a certain capacity in a case of filling the carrier in a column. Therefore, the average particle diameter is preferably 1 to 1,000 μm, more preferably 5 to 500 μm, and still more preferably 10 to 200 μm.

The specific surface area of the Sp carrier, which is measured by a multi-point BET method, is not particularly limited, but in a case where the specific surface area is less than 0.1 m²/g, the swelling degree in an organic solvent is low and thus a synthetic reaction is less likely to occur, and on the other hand, in a case where the specific surface area is more than 500 m²/g, the pore diameter is small and thus a synthetic reaction is less likely to occur. Therefore, the specific surface area is preferably 0.1 to 500 m²/g, more preferably 10 to 300 m²/g, and still more preferably 50 to 200 m²/g.

An average pore diameter of the Sp carrier, which is measured by a mercury intrusion method, is not particularly limited, but in a case where the pore diameter is too small, a reaction field in a synthetic reaction is small and a desired reaction is difficult to occur, or the nucleotides length is less than a desired number, and on the other hand, in a case where the pore diameter is too large, a contact opportunity between a hydroxyl group on the surface of the porous particles, which is a reaction field, and a substance involved in the reaction is small, and thus the yield tends to decrease. Therefore, the average pore diameter is preferably 1 to 200 nm, more preferably 5 to 100 nm, and still more preferably 20 to 70 nm.

### [Carrying Amount]

As described above, the compound (specific carrier) represented by the general formula (3) may be a compound in which one group (residue) obtained by removing Sp from the general formula (3) is bonded to Sp, or may be a compound in which a plurality of the residues are bonded to Sp. An amount (carrying amount) of the residue bonded to Sp is not particularly limited, but in a case where the carrying amount is too small, the yield of the nucleic acid is lowered, and on the other hand, in a case where the carrying amount is too large, the purity of the nucleic acid is lowered. Therefore, the carrying amount is, as the molar amount of the residue with respect to 1 g of Sp, preferably 1 to 2,000 μmol/g, more preferably 10 to 1,000 μmol/g, still more preferably 20 to 100 μmol/g, and particularly preferably 30 to 50 μmol/g.

### [Producing Method]

A method for producing the solid-phase carrier according to the embodiment of the present invention is not particularly limited, and examples thereof include a method of reacting the specific linker with a solid-phase carrier (hereinafter, also referred to as a “reactive solid-phase carrier”) which has a functional group (for example, an amino group) capable of reacting with the carboxy group of the linker and is to be a Sp carrier after the reaction.

Examples of the reactive solid-phase carrier include a CPG solid-phase carrier having a long-chain aminoalkyl spacer (lcaa-CPG solid-phase carrier), a polystyrene-based porous carrier having an amino group and/or a hydroxy group, an acrylamide-based porous carrier having an amino group and/or a hydroxy group (particularly, a hydroxy group), and the like.

### [4] Method for Producing Nucleic Acid

The method for producing a nucleic acid according to the embodiment of the present invention (hereinafter, also simply referred to as “producing method according to the embodiment of the present invention”) is a method for producing a nucleic acid, including a step of performing a nucleic acid synthesis reaction on the solid-phase carrier according to the embodiment of the present invention (specific carrier). That is, the producing method according to the embodiment of the present invention is a method for producing a nucleic acid using a specific carrier as a universal support.

### [Nucleic Acid Synthesis Reaction]

As the nucleic acid synthesis reaction, for example, various known synthesis methods using an automated polynucleotide synthesizer can be used.

It should be noted that, in the present specification, the “nucleic acid synthesis reaction” particularly means an extension reaction of a nucleotide constituting a nucleic acid. That is, an extended oligonucleotide is obtained by sequentially bonding nucleotides to a nucleoside, a nucleotide, or an oligonucleotide, which is bonded to a solid-phase carrier.

In addition, in the present specification, the “nucleic acid” means a chain-like compound (oligonucleotide) in which nucleotides are linked by a phosphodiester bond, and includes DNA, RNA, and the like. The nucleic acid may be either a single-stranded or double-stranded nucleic acid, but is preferably a single-stranded nucleic acid because the nucleic acid can be efficiently synthesized by a polynucleotide synthesizer. In the present specification, the “nucleic acid” includes not only an oligonucleotide containing a purine base such as adenine (A) and guanine (G) and a pyrimidine base such as thymine (T), cytosine (C), and uracil (U), but also a modified oligonucleotide containing these modified nucleic acid bases.

Examples of the nucleic acid synthesis reaction include an H-phosphonate method, a phosphoester method, a phosphoramidite method, and the like, and among these, a phosphoramidite method is preferable because a synthesis ability of nucleic acid is high and high-purity nucleic acid can be obtained.

### [Cutout Treatment]

The producing method according to the embodiment of the present invention usually includes a step (cutout treatment) of cutting out the synthesized nucleic acid from the specific carrier which is a universal support.

The cutout treatment is not particularly limited, and examples thereof include a method of treating with ammonia and/or amines. Examples of the amines include methylamine, ethylamine, isopropylamine, ethylenediamine, diethylamine, triethylamine, and the like. It is desirable that the ammonia and/or the amines are mixed with a solvent to use. Examples of the solvent include water, alcohols (for example, methanol, ethanol, and the like), and the like. Two or more of these solvents may be mixed in appropriate proportions to use.

The nucleic acid is obtained by the cutout treatment. Specific examples of the scheme in which the nucleic acid is cut out from the specific carrier are as described above.

In this case, as described above, in a case where an adduct in which a 1,2-diol unit is added to a nucleic acid remains, in a case of using the solid-phase carrier according to the embodiment of the present invention, the nucleic acid and the adduct can be easily separated by HPLC.

### EXAMPLES

Hereinafter, the present invention will be described in more detail with reference to examples, but the present invention is not limited thereto.

### Example 1

### [Synthesis of Specific Carrier]

PT-CPG (specific carrier) was synthesized as follows.

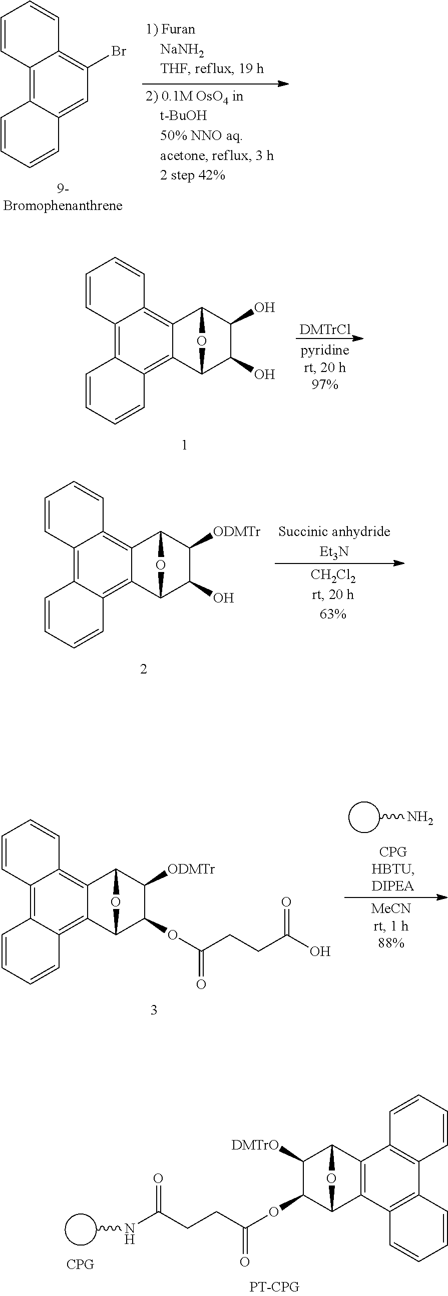

### <Synthesis of Compound 1>

(1) Under an argon stream, furan (5.63 mL, 77.8 mmol) was added to a tetrahydrofuran anhydrous (15 mL) solution of 9-bromophenanthrene (2.00 g, 7.78 mmol) and sodium amide (910 mg, 23.3 mmol) and the mixture was stirred at room temperature for 19 hours. After completion of the reaction, the reaction solution was cooled to 0° C., and water was added thereto. Furthermore, the reaction sodium was diluted with ethyl acetate, an organic layer was washed twice with water and once with saturated saline, and then dried over sodium sulfate, and the solvent was distilled off under reduced pressure. The crude product was purified by silica gel column chromatography (hexane/ethyl acetate=100:1 to 50:1), thereby obtaining a precursor compound of the compound 1 (1.10 g as a mixture) as a light brown solid (Chem. Commun. 2014, 50, 6869-6871).

(2) Subsequently, a 50% N-methylmorpholine oxide aqueous solution (2.28 g, 4.56 mL, 19.5 mmol) and a 0.1 M osmium tetroxide tert-butanol solution (77.8 μL, 0.00778 mmol) were added to an acetone (15 mL) solution of the precursor compound (1.10 g, mixture), and the mixture was heated and refluxed for 3 hours. After completion of the reaction, a sodium thiosulfate-saturated aqueous solution was added to the reaction solution, and extraction was carried out 5 times with a mixed solution of chloroform and methanol (chloroform/methanol=10:1). The collected organic layer was washed once with water and once with saturated saline, and dried over sodium sulfate, and the solvent was distilled off under reduced pressure. The crude product was purified by silica gel chromatography (from only chloroform to chloroform/methanol=100:1), thereby obtaining the compound 1 (compound represented by the general formula (1), where Ar represents a phenanthrene ring, Z represents a hydrogen atom, and R¹ to R⁴ each represent a hydrogen atom) (specific linker precursor) as a light brown solid (910 mg, yield of 42% for 2 steps).

¹H-NMR (500 MHz, DMSO-d₆) δ 8.90-8.88 (m, 2H), 8.10-8.08 (m, 2H), 7.74-7.70 (m, 4H), 5.75 (s, 2H), 5.17-5.15 (m, 2H), 3.70-3.67 (m, 2H). ¹³C-NMR (125 MHz, DMSO-d₆) δ 138.6, 129.7, 127.3, 126.7, 125.6, 124.5, 123.8. IR (ATR) cm⁻¹: 3355, 3240. HRMS (FAB): calcd for C₁₈H₁₄NaO₃ [M+Na]⁺ 301.0841, found 301.0828.

### <Synthesis of Compound 2>

Under an argon stream, 4,4′-dimethoxytrityl chloride (731 mg, 2.16 mmol) was added to a pyridine anhydrous (10 mL) solution of the compound 1 (500 mg, 1.80 mmol) and the mixture was stirred at room temperature for 20 hours. After completion of the reaction, the reaction solution was diluted with ethyl acetate, an organic layer was washed twice with water and once with saturated saline, and then dried over sodium sulfate, and the solvent was distilled off under reduced pressure. The crude product was purified by silica gel column chromatography (hexane/ethyl acetate=3:1), thereby obtaining the compound 2 (compound represented by the general formula (1), where Ar represents a phenanthrene ring, Z represents a DMTr group, and R¹ to R⁴ each represent a hydrogen atom) (specific linker precursor) as a light yellow solid (1.01 g, yield of 97%).

¹H-NMR (500 MHz, CDCl₃) δ 8.66-8.63 (m, 2H), 7.91-7.89 (m, 1H), 7.65-7.48 (m, 6H), 7.39-7.26 (m, 5H), 7.20 (d, 1H, J=7.0 Hz), 6.90-6.86 (m, 4H), 5.73 (s, 1H), 5.12 (s, 1H), 3.98 (d, 1H, J=5.0 Hz), 3.91-3.87 (m, 2H), 3.83 (s, 3H), 3.81 (s, 3H). ¹³C-NMR (125 MHz, CDCl₃) δ 158.9, 158.8, 144.7, 138.4, 138.0, 136.1, 135.8, 130.3, 130.2, 128.3, 128.2, 127.2, 126.7, 126.5, 126.4, 126.0, 125.5, 125.0, 124.4, 123.5, 123.3, 113.5, 88.9, 84.7, 83.0, 73.0, 70.6, 55.3. IR (ATR) cm⁻¹: 3499, 3004, 2953, 1607, 1507. FIRMS (ESI-TOF): calcd for C₃₉H₃₂NaO₅ [M+Na]⁺ 603.2147, found 603.2149.

### <Synthesis of Compound 3>

Under an argon stream, triethylamine (1.19 mL, 8.61 mmol) was added to a dichloromethane anhydrous (10 mL) solution of the compound 2 (500 mg, 0.861 mmol). Succinic anhydride (345 mg, 3.44 mmol) was added thereto, and the mixture was stirred at room temperature for 20 hours. After completion of the reaction, for the reaction solution, extraction was carried out 5 times with a mixed solution of chloroform and methanol (chloroform/methanol=10:1), the collected organic layer was washed once with water and once with saturated saline, and then dried over sodium sulfate, and the solvent was distilled off under reduced pressure. The crude product was purified by silica gel chromatography (from only chloroform to chloroform/methanol=100:1), thereby obtaining the compound 3 (compound represented by the general formula (2), where Ar represents a phenanthrene ring, Z represents a DMTr group, R¹ to R⁴ each represent a hydrogen atom, and L represents an ethylene group) (specific linker) as a light brown solid (368 mg, yield of 63%).

¹H-NMR (500 MHz, CDCl₃) δ 8.63 (d, 2H, J=8.5 Hz), 7.95-7.93 (m, 1H), 7.64-7.60 (m, 3H), 7.55-7.49 (m, 3H), 7.42-7.36 (m, 4H), 7.31-7.21 (m, 4H), 6.87-6.82 (m, 4H), 5.83 (s, 1H), 5.26 (s, 1H), 4.76 (d, 1H, J=6.0 Hz), 4.09 (d, 1H, J=6.0 Hz), 3.83 (s, 3H), 3.81 (s, 3H), 2.95-2.77 (m, 4H). ¹³C-NMR (125 MHz, CDCl₃) δ 172.7, 158.7, 158.6, 145.4, 138.7, 137.8, 136.7, 136.4, 130.3, 130.2, 128.3, 128.0, 127.4, 126.9, 126.8, 126.7, 126.4, 125.8, 125.4, 125.0, 123.4, 123.3, 113.4, 88.1, 84.7, 83.6, 82.6, 73.9, 73.1, 55.3, 29.3. IR (ATR) cm⁻¹: 3649, 3007, 2929, 1733, 1607, 1507. HRMS (ESI-TOF): calcd for C₄₃H₃₆NaO₈ [M+Na]⁺ 703.2308, found 703.2303.

### <Synthesis of PT-CPG>

Under an argon stream, diisopropylethylamine (6.80 μL, 40.0 μmol) was added to an acetonitrile anhydrous (4 mL) suspension of lcaa (long-chain aminoalkyl spacer)-CPG (500 mg, amino group content: 40.0 μmol/g), and the compound 3 (13.6 mg, 20.0 μmol) and HBTU (Hexafluorophosphate Benzotriazole Tetramethyl Uronium) (7.58 mg, 20.0 μmol) were added thereto. The reaction solution was stirred at room temperature for 1 hour, and then filtered, and the solid-phase carrier (filtered product) was washed with acetonitrile and further dried in vacuum for overnight. The obtained solid was suspended in a Cap A solution (tetrahydrofuran solution of acetic acid anhydride) and a Cap B solution (tetrahydrofuran solution of methylimidazole) and stirred for 30 minutes. The reaction solution was filtered again, and the solid-phase carrier (filtered product) was washed with acetonitrile and dried in vacuum for overnight, thereby obtaining PT-CPG (compound represented by the general formula (3), where Ar represents a phenanthrene ring, Z represents a DMTr group, R¹ to R⁴ each represent a hydrogen atom, L represents an ethylene group, and Sp represents a carrier obtained by removing an amino group from a long-chain aminoalkyl spacer of lcaa-CPG) (specific carrier). The carrying amount of the compound 3 in PT-CPG was quantified by a DMTr assay method and was 37±1.3 μmol/g. The DMTr assay method is a method of indirectly quantifying the carrying amount by treating a solid-phase carrier with a deblocking solution (dichloromethane solution of 3 w/v % trichloroacetic acid) and measuring the amount of deprotected dimethoxytrityl (DMTr) group by absorbance measurement (504 nm).

### [Synthesis of Oligonucleic Acid]

Using PT-CPG as a universal support, an oligonucleic acid (T₁₀) of T-10mer was synthesized by an automated DNA synthesizer (in the formulae, T represents a thymine residue and CE represents a cyanoethyl group). The synthesis scale was 0.2 μmol, and the synthesis was carried out under the trityl OFF condition (finally, the DMTr group was deprotected by a dichloromethane solution of 3 w/v % trichloroacetic acid). The commercially available phosphoramidite as T was prepared as a 0.1 M acetonitrile anhydrous solution to use. As an activation agent, 5-ethylthio-1H-tetrazole (0.25 M acetonitrile anhydrous solution) was used, and the condensation time was 10 minutes only for the 3′ terminal and 25 seconds for the other portions.

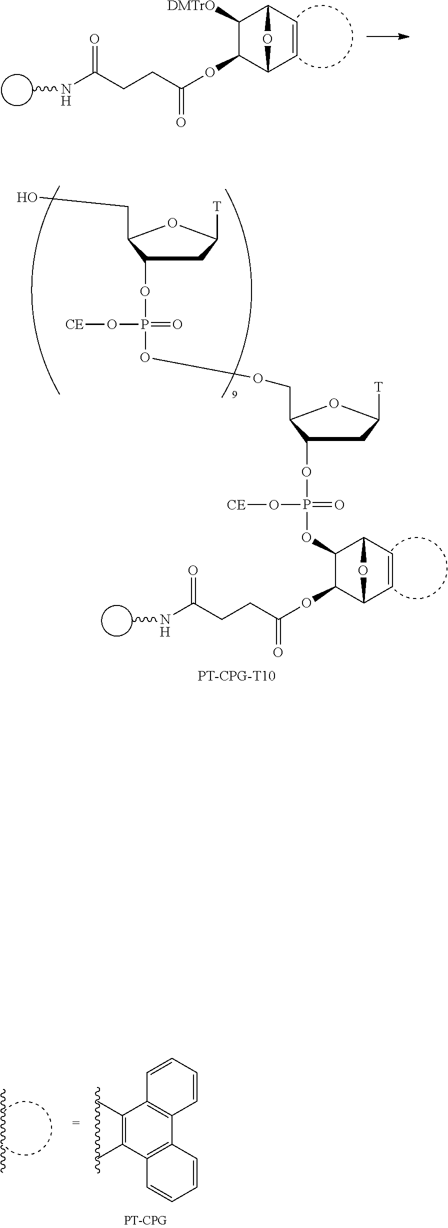

[Cutting Out of Oligonucleic Acid from Universal Support and Removal of Linker]

The cutting out of the oligonucleic acid from the universal support and the removal of the linker associated therewith were performed by treating with a 28% ammonia aqueous solution at room temperature for 2 hours (condition A).

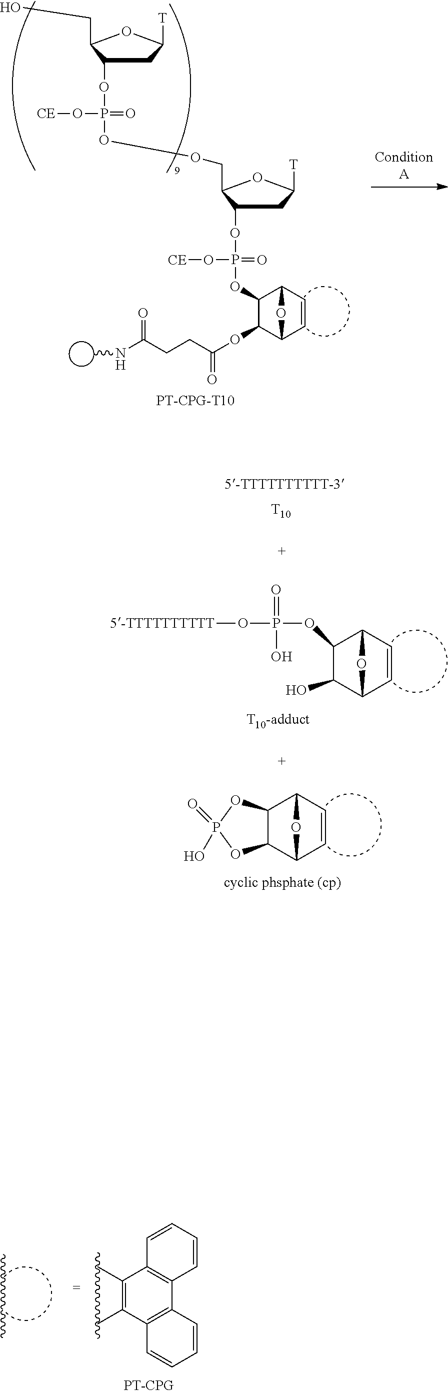

### Comparative Example 1

An oligonucleic acid was synthesized and subjected to a cutout treatment in the same manner as in Example 1, except that CUTAG-CPG (manufactured by Sigma-Aldrich Co. LLC) (the following structure, R: methyl group) (hereinafter, also simply referred to as “CUTAG”) was used instead of PT-CPG.

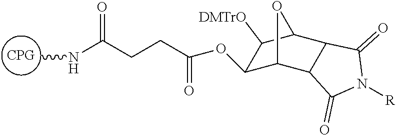

### [Evaluation]

In Examples 1 and Comparative Example 1, the solution after the cutout treatment was analyzed by reversed-phase HPLC.

### [Condition 1]

First, HPLC was carried out under the conditions shown in Table 1 below.

TABLE 1

| Eluate | A liquid: 0.1M triethylammonium acetate | |
|---|---|---|
| | buffer solution (pH 7.0) | |
| | B liquid: acetonitrile | |
| Gradient | B liquid: 5% to 15% (30 minutes) | |
| Column | Waters Xbridge ™ Shield RP18 2.5 μm (4.6 × 50 mm) | |
| Flow rate | 1.0 | mL/minute |
| Column | 40° | C. |
| temperature | | |

| | |
|---|---|
| Detection | UV (254 nm) |

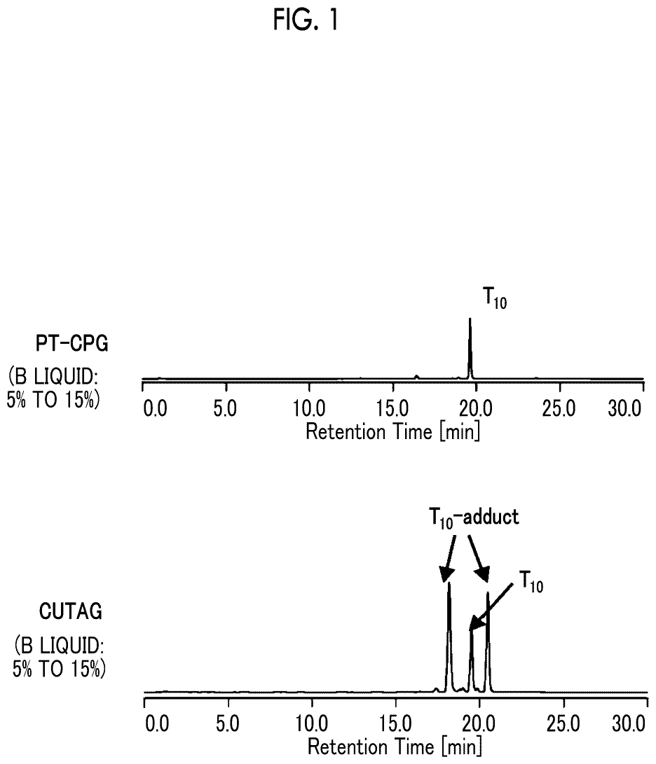

FIG. 1 shows a chart of HPLC.

As shown in FIG. 1, in Comparative Example 1, peaks of the oligonucleic acid (T₁₀) and the adduct (T₁₀-aduct) were observed. On the other hand, in Example 1, only the peak of the oligonucleic acid was observed.

### [Condition 2]

The reason why the peak of the adduct was not observed in Example 1 was presumed to be that the retention time of the peak of the adduct was long. Therefore, in Example 1, HPLC was measured under more difficult separation conditions. Specifically, the gradient was changed from “B solution: 5% to 15%” to “B solution: 8% to 18%”. The conditions of HPLC are shown in Table 2 below.

TABLE 2

| Eluate | A liquid: 0.1M triethylammonium acetate | |
|---|---|---|
| | buffer solution (pH 7.0) | |
| | B liquid: acetonitrile | |
| Gradient | B liquid: 5% to 18% (30 minutes) | |
| Column | Waters Xbridge ™ Shield RP18 2.5 μm (4.6 × 50 mm) | |
| Flow rate | 1.0 | mL/minute |
| Column | 40° | C. |
| temperature | | |

| | |
|---|---|
| Detection | UV (254 nm) |

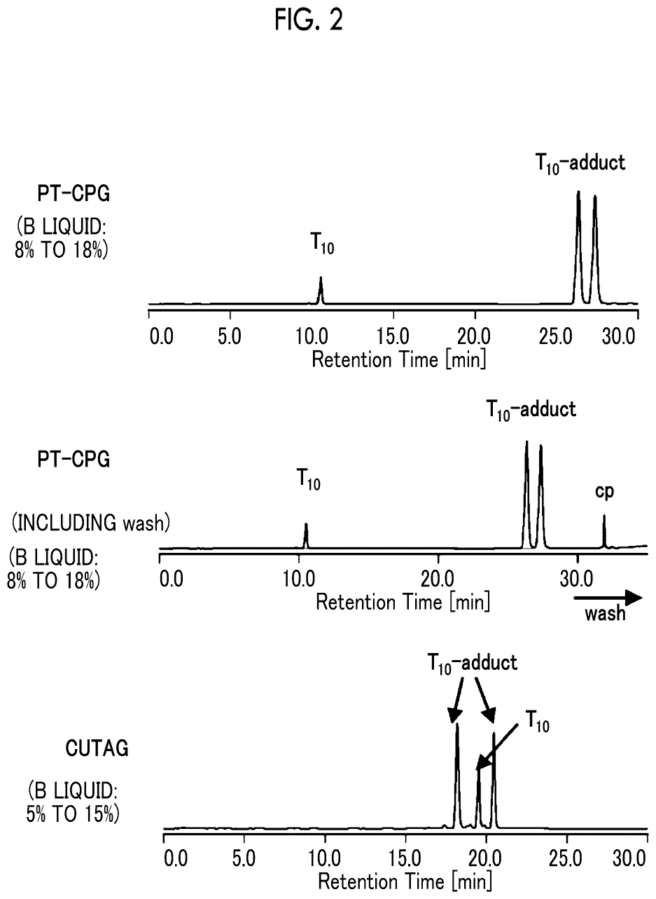

FIG. 2 shows a chart of HPLC. In addition, a chart (including wash) including the period of the washing (30 minutes or more) is also shown together. In addition, for comparison, the chart of Comparative Example 1 (B liquid: 5% to 15%) is also shown together.

### [Result]

As shown in FIG. 1, in a case of Comparative Example 1, the peak of the oligonucleic acid (T₁₀) and the peak of the adduct (T₁₀-adduct) were observed at close positions. On the other hand, as shown in FIG. 2, in the case of Example 1, even under the condition (B liquid: 8% to 18%) where the separation was more difficult, the peak of the oligonucleic acid and the peak of the adduct were sufficiently separated. That is, in a case where a nucleic acid was synthesized using the specific carrier of Example 1, it was shown that the nucleic acid and the adduct could be easily separated by HPLC in a case where the nucleic acid was cut out. In addition, in the case of Example 1, a peak of a cyclic phosphate (cp) derived from the linker was also observed in the HPLC chart (including wash).

[Comparison with Tetrahedron, 2021, 92, 132261]

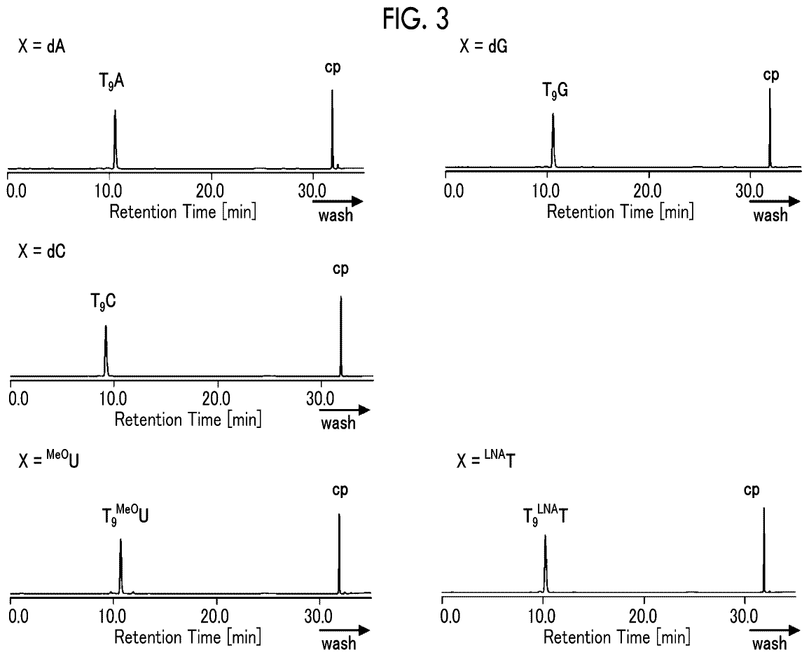

In CPG3 of FIG. 3 of Tetrahedron, 2021, 92, 132261, a chart of HPLC (the same conditions as those in the condition 2) of a solution after the cutout treatment in a case where an oligonucleic acid (T₁₀) was synthesized using a universal support obtained from the following linker was shown, and a peak of the oligonucleic acid (T₁₀) was observed at a retention time of about 10 minutes and a peak of the adduct (T₁₀-3) was observed at a retention time of about 24 minutes.

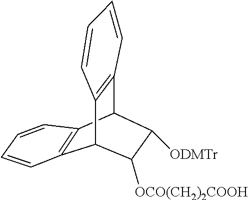

On the other hand, in the case of Example 1, as shown in FIG. 2, the peak of the oligonucleic acid (T₁₀) was observed at a retention time of about 10 minutes, and the peak of the adduct (T₁₀-adduct) was observed at a retention time of about 27 minutes. That is, it can be said that the specific carrier of Example 1 is more excellent in the separation of the peak than the universal support of Tetrahedron, 2021, 92, 132261.

### Example 2

### [Synthesis of Oligonucleic Acid]

Using PT-CPG as a universal support, an oligonucleic acid (T₉X) in which a nucleoside other than T was introduced only at the 3′ terminal was synthesized by an automated DNA synthesizer. As the 3′ terminal (X), PAc-dA (dA), Ac-dC (dC), iPrPac-dG (dG), 2′-O-Me-U (^{MeO}U), and LNA-T (^{LNA}T) were used. The synthesis scale was 0.2 μmol, and the synthesis was performed under the trityl OFF condition. The commercially available phosphoramidite as T was prepared as a 0.1 M acetonitrile anhydrous solution to use. As an activation agent, 5-ethylthio-1H-tetrazole (0.25 M acetonitrile anhydrous solution) was used, and the condensation time was 10 minutes only for the 3′ terminal and 25 seconds for the other portions.

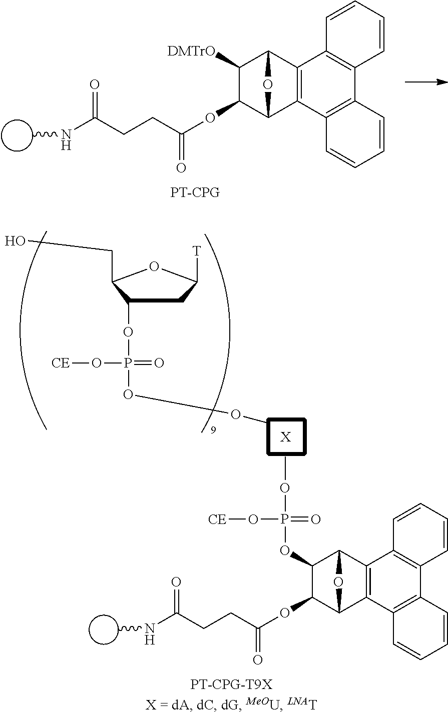

[Cutting Out of Oligonucleic Acid from Universal Support and Removal of Linker]

The cutting out of the oligonucleic acid from the universal support and the removal of the linker associated therewith were performed by treating with a 28% ammonia aqueous solution at 55° C. for 8 hours (condition B).

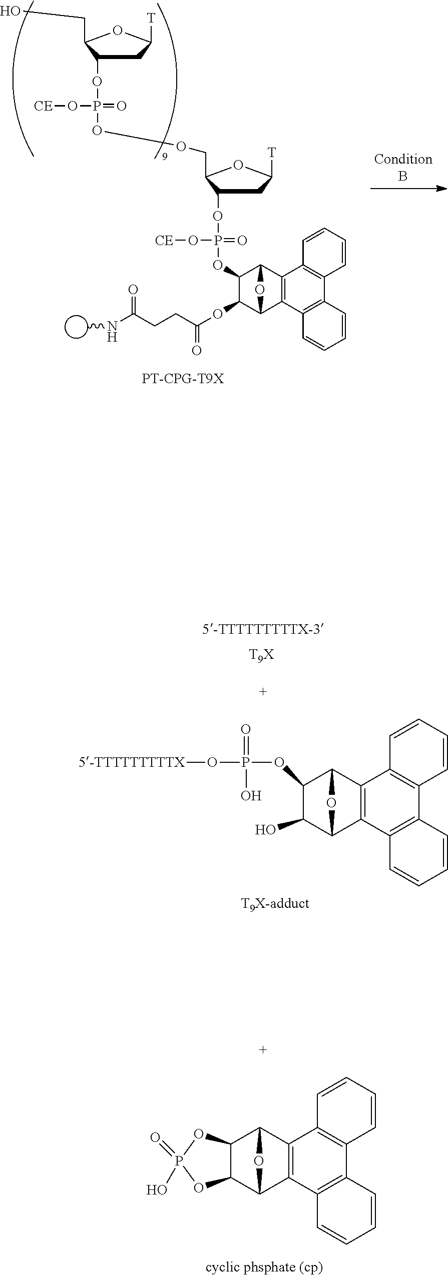

### [Evaluation]

The solution after the cutout treatment was analyzed by reversed-phase HPLC. The conditions of HPLC were the same as those of the condition 2.

FIG. 3 shows a chart of HPLC.

As shown in FIG. 3, a peak of the oligonucleic acid (T₉X) and a peak of the cyclic phosphate (cp) derived from the linker were observed. It should be noted that, even though the analysis was performed under the same conditions (condition 2) as in FIG. 2, the peak of the adduct (T₉X-adduct) was not observed. From this, it is considered that the oligonucleic acid is completely cut out and no adduct exists in the solution after the cutout treatment.

### Example 3

### [Synthesis of Oligonucleic Acid]

Using PT-CPG as a universal support, a phosphorothioate-modified oligonucleic acid (sON) was synthesized by an automated DNA synthesizer. The synthesis scale was 0.2 μmol, and the synthesis was performed under the trityl OFF condition. Commercially available T, ^{Ac}C, ^{Pac}A, and ^{iPrPac}G phosphoramidites were prepared as 0.1 M acetonitrile anhydrous solution to use. As an activation agent, 5-ethylthio-1H-tetrazole (0.25 M acetonitrile anhydrous solution) was used, and the condensation time was 10 minutes only for the 3′ terminal and 25 seconds for the other portions. As a sulfurizing agent, ((dimethylamino-methylidene)amino)-3H-1,2,4-dithiazaoline-3-thione (DDTT) (0.05 M pyridine anhydrous/acetonitrile anhydrous solution) was used.

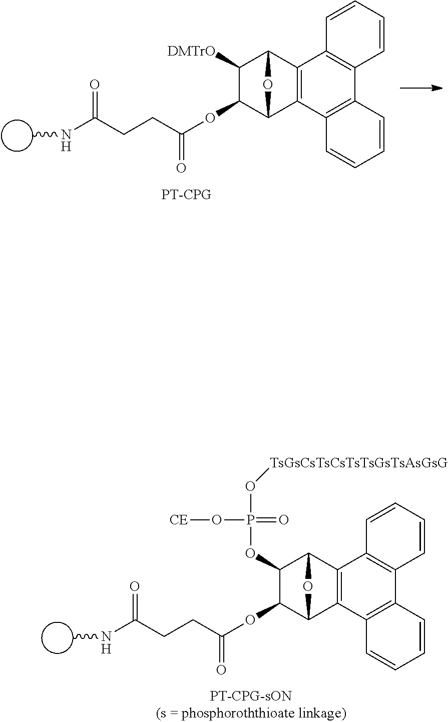

[Cutting Out of Oligonucleic Acid from Universal Support and Removal of Linker]

The cutting out of the oligonucleic acid from the universal support and the removal of the linker associated therewith were performed by treating with a 28% ammonia aqueous solution at room temperature for 2 hours (condition A) or treating with a 28% ammonia aqueous solution at 55° C. for 3 ours (condition C).

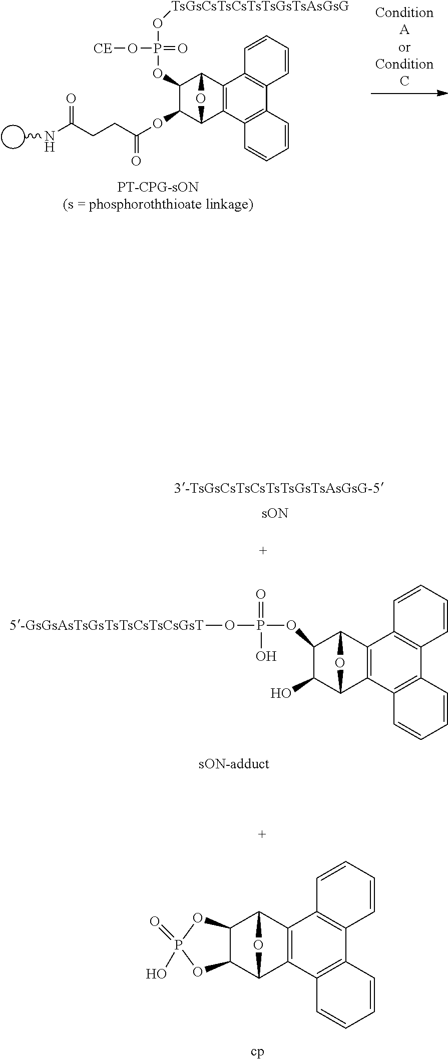

### [Evaluation]

The solution after the cutout treatment was analyzed by reversed-phase HPLC. The conditions of HPLC are as shown in Table 3 below (condition 3).

TABLE 3

| Eluate | A liquid: 0.1M triethylammonium acetate | |
|---|---|---|
| | buffer solution (pH 7.0) | |
| | B liquid: acetonitrile | |
| Gradient | B liquid: 5% to 25% (30 minutes) | |
| Column | Waters Xbridge ™ Shield RP18 2.5 μm (4.6 × 50 mm) | |
| Flow rate | 1.0 | mL/minute |
| Column | 40° | C. |
| temperature | | |

| | |
|---|---|
| Detection | UV (254 nm) |

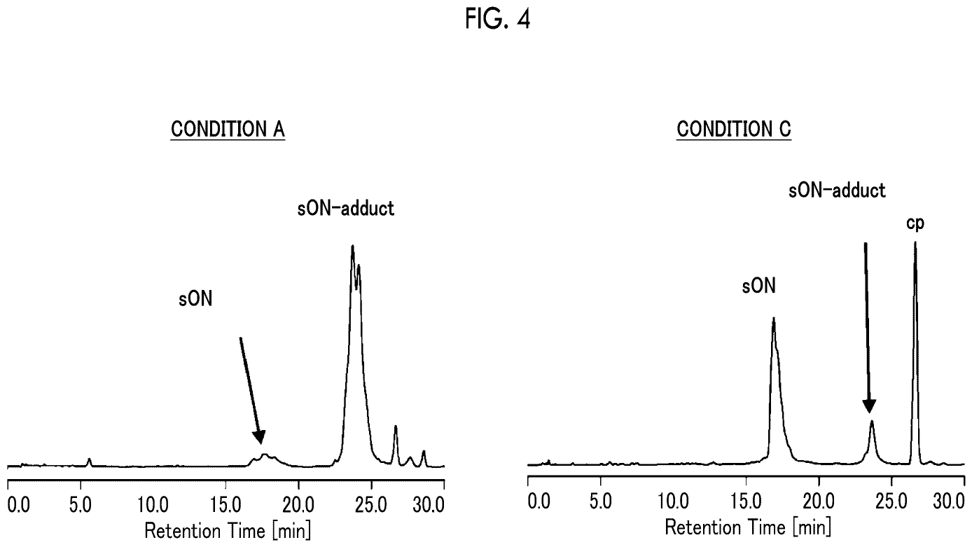

FIG. 4 shows a chart of HPLC.

As shown in FIG. 4, the peak of the phosphorothioate-modified oligonucleic acid (sON) and the peak of the adduct (sON-adduct) were separated from each other. In addition, in the HPLC chart under the condition C, a peak of a cyclic phosphate (cp) derived from the linker was also observed.

### Example 4

### [Synthesis of Specific Carrier]

PT-PS (a) (specific carrier) and PT-PS (c) (specific carrier) were synthesized as follows.

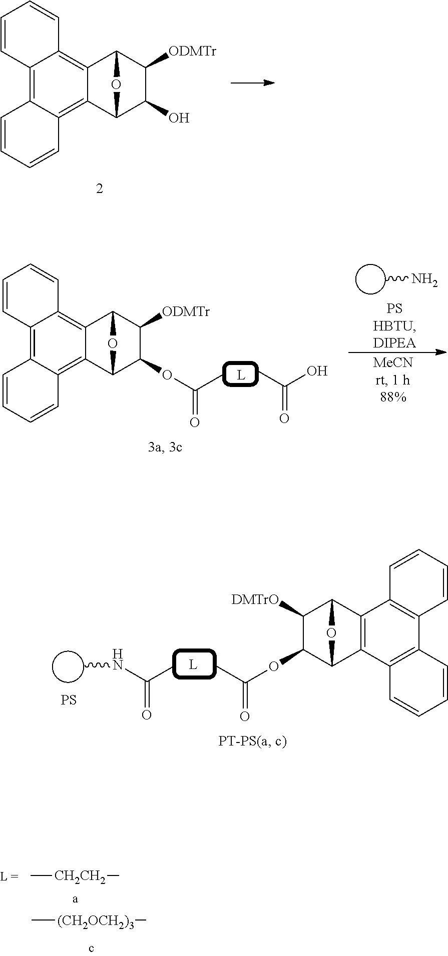

<PT-PS (a)>

Under an argon stream, diisopropylethylamine (30.8 μL, 180 μmol) was added to an acetonitrile anhydrous (4 mL) suspension of Primer Support™ (200 mg, amino group content: 90.0 μmol), and compound 3a (61.3 mg, 90.0 μmol) which was synthesized by the same procedure as in Compound 3 of Example 1 and HBTU (34.1 mg, 90.0 μmol) were further added thereto. The reaction solution was stirred at room temperature for 1 hour, and then filtered, and the solid-phase carrier (filtered product) was washed with acetonitrile and further dried in vacuum for overnight. The obtained solid was suspended in a Cap A solution (acetonitrile solution of acetic acid anhydride) and a Cap B solution (acetonitrile solution of methylimidazole) and stirred for 30 minutes. The reaction solution was filtered again, and the solid-phase carrier (filtered product) was washed with acetonitrile and dried in vacuum for overnight, thereby obtaining PT-PS (a). The carrying amount of the compound 3a of PT-PS (a) was quantified by a DMTr assay method and was 349±10 μmol/g.

<PT-PS (c)>

### (Synthesis of Compound 3c)

Under an argon stream, 2,2′-((oxybis(ethane-2,1-diyl))bis(oxy))diacetic acid (91.8 mg, 0.413 mmol) was added to a pyridine anhydrous (3 mL) solution of the compound 2 (200 mg, 0.344 mmol) synthesized by the same procedure as in Example 1. 1-Ethyl-3-(3-dimethylaminopropyl)carbodiimide hydrochloride (65.9 mg, 0.344 mmol) and 4-dimethylaminopyridine (4.20 mg, 0.00344 mmol) were added thereto, and the mixture was stirred at room temperature for 20 hours. After completion of the reaction, extraction was carried out 5 times with a mixed solution of chloroform and methanol (chloroform/methanol=10:1). The collected organic layer was washed once with water and once with saturated saline, and dried over sodium sulfate, and the solvent was distilled off under reduced pressure. The crude product was purified by silica gel chromatography (from only chloroform to chloroform/methanol=20:1), thereby obtaining the compound 3c as a white solid (105 mg, yield of 39%).

¹H-NMR (500 MHz, CDCl₃) δ: 8.66-8.64 (m, 2H), 7.97-7.95 (m, 1H), 7.66-7.61 (m, 3H), 7.57-7.54 (m, 1H), 7.48-7.47 (m, 2H), 7.47-7.22 (m, 8H), 6.87-6.83 (m, 4H), 5.87 (s, 1H), 5.33 (s, 1H), 4.79 (d, 1H, J=6.0 Hz), 4.48 and 4.24 (ABq, 1H, 1H, J=16.5 Hz), 4.16 (s, 2H), 4.12 (d, 1H, J=6.0 Hz), 3.86-3.72 (m, 14H). ¹³C-NMR (125 MHz, CDCl₃) δ: 171.4, 171.0, 158.7, 145.3, 138.7, 137.5, 136.6, 136.2, 130.4, 130.3, 128.3, 128.0, 127.0, 126.8, 126.5, 125.8, 125.3, 125.0, 124.5, 123.4, 113.4, 88.1, 83.6, 82.6, 77.3, 77.2, 77.0, 76.8, 73.9, 73.1, 71.5, 70.8, 70.7, 70.3, 69.0, 68.6, 55.3, 45.1, 9.2. IR (ATR) cm⁻¹: 2929, 1746, 1607, 1507. HRMS (ESI-TOF): calcd for C₄₇H₄₃O₁₁ [M-H]^{?} 783.2805, found 783.2809.

### (Synthesis of PT-PS (c))

Under an argon stream, diisopropylethylamine (15.4 μL, 90.0 μmol) was added to an acetonitrile anhydrous (2 mL) suspension of Primer Support™ (100 mg, amino group content: 45.0 μmol), and compound 3c (35.3 mg, 45.0 μmol) and HBTU (17.1 mg, 45.0 μmol) were further added thereto. The reaction solution was stirred at room temperature for 1 hour, and then filtered, and the solid-phase carrier (filtered product) was washed with acetonitrile and further dried in vacuum for overnight. The obtained solid was suspended in a Cap A solution (acetonitrile solution of acetic acid anhydride) and a Cap B solution (acetonitrile solution of methylimidazole) and stirred for 30 minutes. The reaction solution was filtered again, and the solid-phase carrier (filtered product) was washed with acetonitrile and dried in vacuum for overnight, thereby obtaining PT-PS (c). The carrying amount of the compound 3c of PT-PS (c) was quantified by a DMTr assay method* and was 296±21 μmol/g.

### [Synthesis of Oligonucleic Acid]

PT-PS (a) and PT-PS (c) were used as a universal support, and an oligonucleic acid (T₁₀) of T-10mer was synthesized by an automated DNA synthesizer. The synthesis scale was 1.0 μmol, and the synthesis was performed under the trityl OFF condition. The commercially available phosphoramidite as T was prepared as a 0.1 M acetonitrile anhydrous solution to use. As an activation agent, 5-ethylthio-1H-tetrazole (0.25 M acetonitrile anhydrous solution) was used, and the condensation time was 10 minutes only for the 3′ terminal and 25 seconds for the other portions.

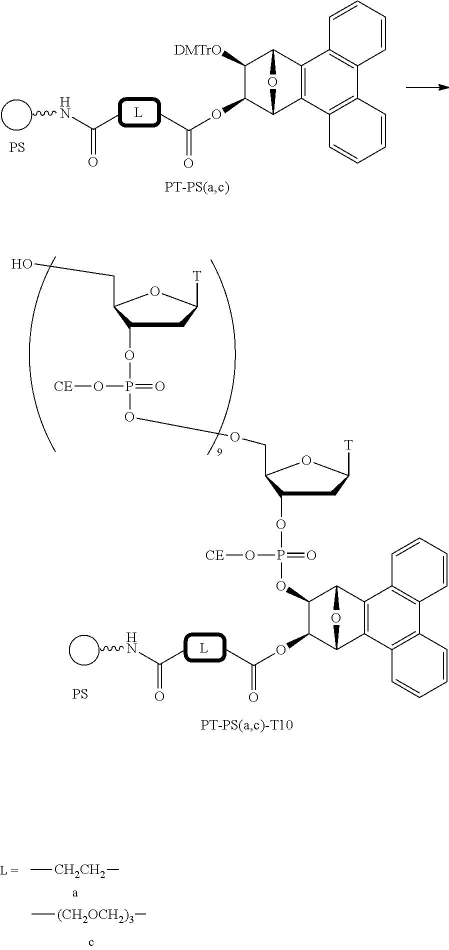

[Cutting Out of Oligonucleic Acid from Universal Support and Removal of Linker]

The cutting out of the oligonucleic acid from the universal support and the removal of the linker associated therewith were performed by treating with a 28% ammonia aqueous solution at 55° C. for 48 hours.

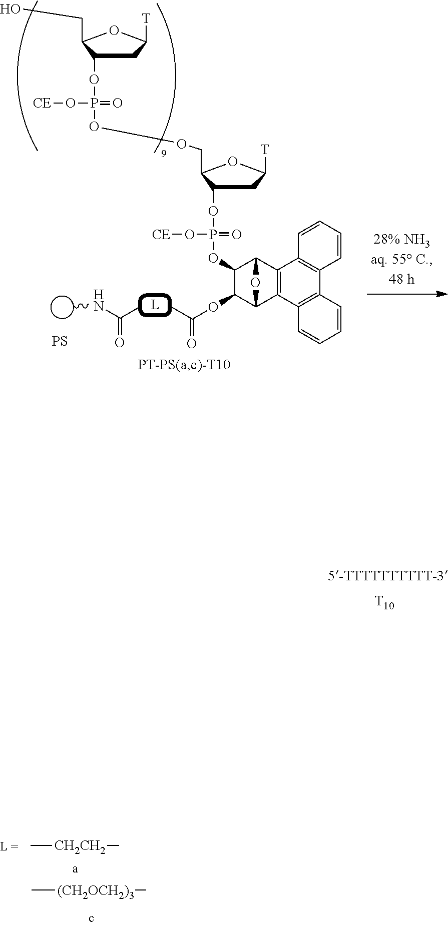

### [Evaluation]

The solution after the cutout treatment was analyzed by reversed-phase HPLC. The conditions of HPLC were the same as those of the condition 2.

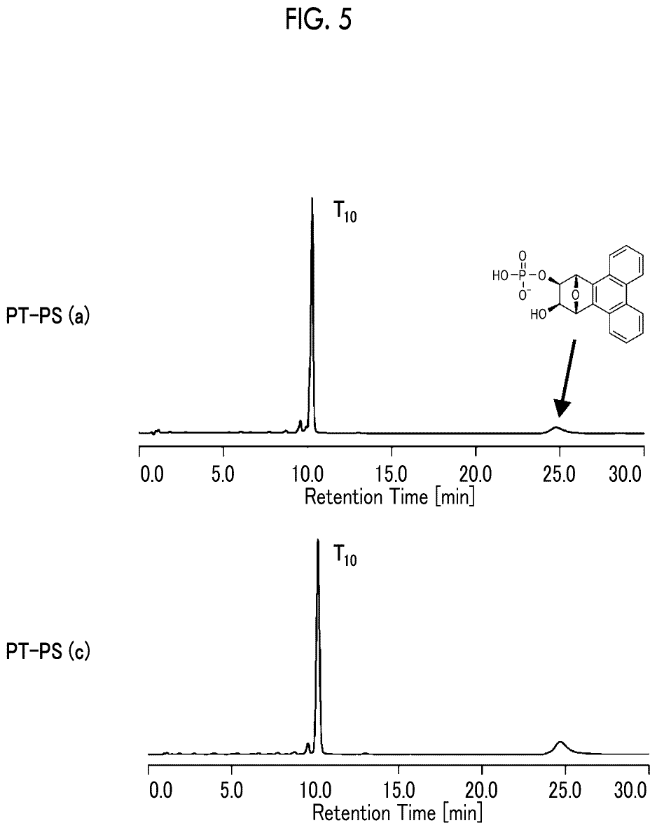

FIG. 5 shows an HPLC chart.

As shown in FIG. 5, a peak of the oligonucleic acid (T₁₀) was observed.

1. A compound represented by the following general formula (1), 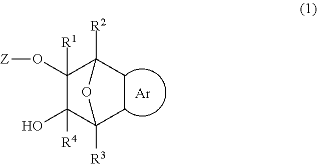
in the general formula (1), Ar represents a tri- to tetracyclic aromatic ring which may have a substituent, Z represents a hydrogen atom or a protective group eliminable by an acid, and R¹ to R⁴ each independently represent a hydrogen atom, an alkyl group, or an alkoxy group.

2. A compound represented by the following general formula (2), 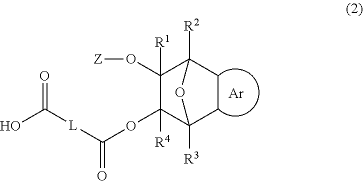
in the general formula (2), Ar represents a tri- to tetracyclic aromatic ring which may have a substituent, Z represents a hydrogen atom or a protective group eliminable by an acid, R¹ to R⁴ each independently represent a hydrogen atom, an alkyl group, or an alkoxy group, and L represents a divalent hydrocarbon group which may have an oxygen atom.

3. The compound according to claim 1,
wherein Ar represents an anthracene ring, a phenanthrene ring, a tetracene ring, or a pyrene ring, each of which may have a substituent.

4. The compound according to claim 1,
wherein Ar represents a phenanthrene ring which may have a substituent.

5. The compound according to claim 1,
wherein Z represents a protective group eliminable by an acid.

6. The compound according to claim 5,
wherein the protective group eliminable by an acid is a trityl-based protective group or a silyl-based protective group.

7. The compound according to claim 1,
wherein R¹ to R⁴ each represent a hydrogen atom.

8. The compound according to claim 2,
wherein L represents an alkylene group which may have an oxygen atom, an arylene group which may have an oxygen atom, or a combination of the alkylene group and the arylene group.

9. A linker of a solid-phase carrier for nucleic acid synthesis, obtained by using the compound according to claim 1.

10. A solid-phase carrier formed of a compound represented by the following general formula (3), 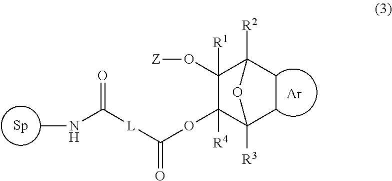
in the general formula (3), Ar represents a tri- to tetracyclic aromatic ring which may have a substituent, Z represents a hydrogen atom or a protective group eliminable by an acid, R¹ to R⁴ each independently represent a hydrogen atom, an alkyl group, or an alkoxy group, L represents a divalent hydrocarbon group, and Sp represents a solid-phase carrier.

11. The solid-phase carrier according to claim 10, wherein the solid-phase carrier is for nucleic acid synthesis.

12. The solid-phase carrier according to claim 10,
wherein Ar represents an anthracene ring, a phenanthrene ring, a tetracene ring, or a pyrene ring, each of which may have a substituent.

13. The solid-phase carrier according to claim 10,
wherein Ar represents a phenanthrene ring which may have a substituent.

14. The solid-phase carrier according to claim 10,
wherein Z represents a protective group eliminable by an acid.

15. The solid-phase carrier according to claim 14,
wherein the protective group eliminable by an acid is a trityl-based protective group or a silyl-based protective group.

16. The solid-phase carrier according to claim 10,
wherein R¹ to R⁴ each represent a hydrogen atom.

17. The solid-phase carrier according to claim 10,
wherein L represents an alkylene group which may have an oxygen atom, an arylene group which may have an oxygen atom, or a combination of the alkylene group and the arylene group.

18. The solid-phase carrier according to claim 10,
wherein Sp represents a porous polymer carrier or a glass-based porous carrier.

19. A method for producing a nucleic acid, comprising:
a step of performing a nucleic acid synthesis reaction on the solid-phase carrier according to claim 10.

20. The method for producing a nucleic acid according to claim 19,
wherein the nucleic acid synthesis reaction is performed by a phosphoramidite method.
